(19) United States
(12) Patent Application Publication
(10) Pub. No.: US 2025/0000674 A1
(43) Pub. Date: Jan. 2, 2025

(54) KNEE JOINT FOR PROSTHETIC LEG

(71) Applicant: Seamless Transition LLC, Arlington, VA (US)

(72) Inventors: Sarah N. Malinowski, Arlington, VA (US); Daniel T. Malinowshi, Henderson, MN (US)

(73) Assignee: Seamless Transition LLC, Arlington, VA (US)

(21) Appl. No.: 18/761,262

(22) Filed: Jul. 1, 2024

Related U.S. Application Data
(60) Provisional application No. 63/524,032, filed on Jun. 29, 2023.

(51) Int. Cl. A61F 2/64; A61F 2/50; A61F 2/70; A61F 2/76

(52) U.S. Cl. CPC A61F 2/644; A61F 2002/5003; A61F 2002/5083; A61F 2002/701; A61F 2002/704; A61F 2002/764

(57) ABSTRACT

A prosthetic knee unit useful with a prosthetic leg. The prosthetic knee unit includes a linkage assembly, at least one motor module, and a controller. The linkage assembly includes a base, a driven shaft at least one link, and an output body. The motor module is operably connected to the driven shaft. The controller is programmed to prompt operation of the at least one motor module. Operation of the at least one motor module, as prompted by the controller, articulates the output body relative to the base via pivoting movement of the at least one link. In some embodiments, the prosthetic knee unit further includes at least one sensor, such as an IMU worn by the user. With these and related embodiments, the controller is programmed to receive sensed information from the sensor and to control operation of the at least one motor module based upon the sensed information.

### CROSS-REFERENCE TO RELATED APPLICATIONS

This Non-Provisional patent application claims the benefit of the filing date of U.S. Provisional Patent Application No. 63/524,032, filed Jun. 29, 2023, the entire teachings of which are incorporated herein by reference.

### BACKGROUND

Prosthetic limbs are commonly used to replace missing or non-functional limbs due to injury, disease or other conditions. Prosthetic legs, in particular, are used to provide support and mobility for individuals who do not have one or both legs. The knee joint is an important component of a prosthetic leg as it provides the necessary flexion and extension required for walking and other activities.

The present disclosure addresses problems and limitations associated with the related art.

### SUMMARY

Some aspects of the present disclosure are directed to a prosthetic knee unit useful with a prosthetic leg. The prosthetic knee unit includes a linkage assembly, at least one motor module, and a controller. The linkage assembly includes a base, a driven shaft at least one link, and an output body. The motor module is operable connected to the driven shaft. The controller is programmed to prompt operation of the at least one motor module. With this in mind, the prosthetic knee unit is configured such that the operation of the at least one motor module, as prompted by the controller, articulates the output body relative to the base via pivoting movement of the at least one link. In some embodiments, the at least one motor module is operable as an actuator and as a damper relative to the linkage assembly. In some embodiments, the prosthetic knee unit further includes at least one sensor, such as an inertial measurement unit (IMU) worn by the user. With these and related embodiments, the controller is programmed to receive sensed information from the sensor and to control operation of the at least one motor module.

### BRIEF DESCRIPTION OF THE DRAWINGS

FIG. 1A is a front perspective view of a prosthetic leg in accordance with principles of the present disclosure and represent use thereof relative to a simplified representation of a portion of a human anatomy;

FIG. 1B is a rear perspective view of the arrangement of FIG. 1A;

FIG. 2A is a rear perspective view of a linkage assembly and motor modules in accordance with principles of the present disclosure and useful with the prosthetic leg of FIG. 1A, with the linkage assembly shown in an upright arrangement;

FIG. 2B is a front perspective view of the arrangement of FIG. 2A;

FIG. 2C is a side plan view of the arrangement of FIG. 2A;

FIG. 2D is a longitudinal cross-sectional view of the arrangement of FIG. 2A;

FIG. 3A is a rear perspective view of the linkage assembly and motor modules of FIG. 2A, with the linkage assembly shown in a bent arrangement;

FIG. 3B is a front perspective view of the arrangement of FIG. 3A;

FIG. 3C is a side plan view of the arrangement of FIG. 3A; and

FIG. 3D is a longitudinal cross-sectional view of the arrangement of FIG. 3A.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Traditional prosthetic knees have been limited in their ability to replicate the natural movement and flexibility of a human knee joint. Many prosthetic knees only allow for a limited range of motion, which can make it difficult for users to perform everyday activities, such as walking up and down stairs or engaging in sports and other physical activities. Various examples of the disclosure are designed to achieve biomimicry of human knee movements and accommodate size variations of the joint, providing the transfemoral amputee the ability to both walk and run on the same prosthetic. Examples of the disclosure are capable of natural knee movement. Various examples of the disclosure include one or pneumatic actuators operating a linkage assembly to aid in knee flexion at high speeds and rapid changes in speed. Examples of the disclosure allow the user to move throughout day without constantly considering their prosthetic; its settings, programming, or capabilities. Ultimately, various examples of the disclosure will give transfemoral amputees a renewed ability to move freely.

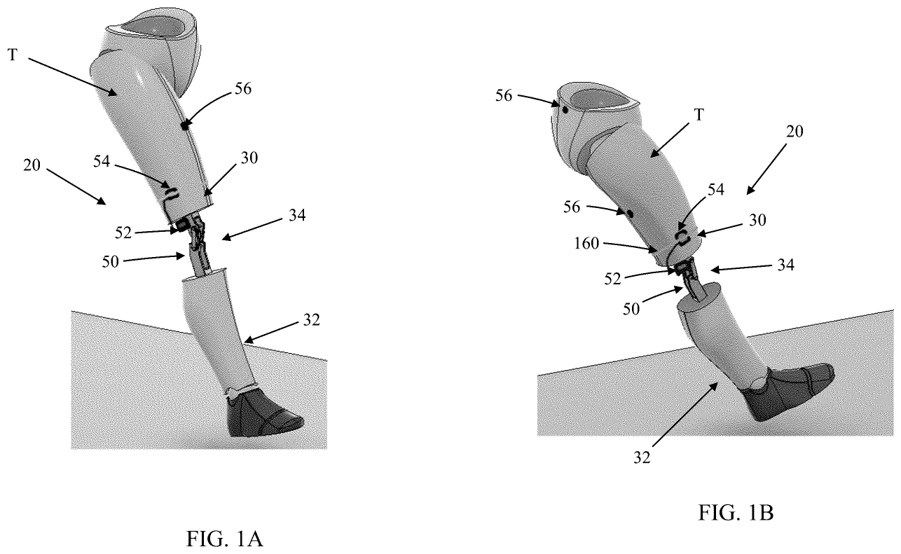

Referring to FIGS. 1A and 1B, in one example, a prosthetic leg 20 in accordance with principles of the present disclosure include a socket 30, a prosthetic lower leg assembly 32, and a prosthetic knee unit 34. The socket 30 is represented in highly simplified form in FIGS. 1A and 1B, and can assume a variety of forms known in the art. In general terms, the socket 30 is generally configured to receive a residual limb of a human (e.g., a portion of a residual thigh T). Similarly, the prosthetic lower leg assembly 32 can assume a wide variety of forms known in the art. In general terms, the prosthetic lower leg assembly 32 includes one or more bodies or components akin to, and serving as, a human calf/lower leg 40 and a foot 42. In some examples, the prosthetic lower leg assembly 32 can be or include a pylon-type construction as is known in the art that can form a curved blade configured for running and the like.

The prosthetic knee unit 34 includes a linkage assembly 50, one or more motor modules 52, a controller 54, and one or more sensor 56. Details on the various components are provided below. In general terms, the linkage assembly 50 is disposed between the socket 30 and the prosthetic lower leg assembly 32, and is configured to facilitate articulation of the prosthetic lower leg assembly 32 relative to the socket 30 (and thus relative to the residual thigh T) akin to a native human knee joint. The motor module(s) 52 provide an input to the linkage assembly 50, and operate to control articulation of the linkage assembly 50 in a determined manner. The controller 54 is configured to prompt operation of the motor(s) 52 (and thus movement of the linkage assembly 50) based on programming. In this regard, the programming operated by the controller 54 utilizes information from the one or more sensors 56 to in dictating operation of the motor module(s) 52.

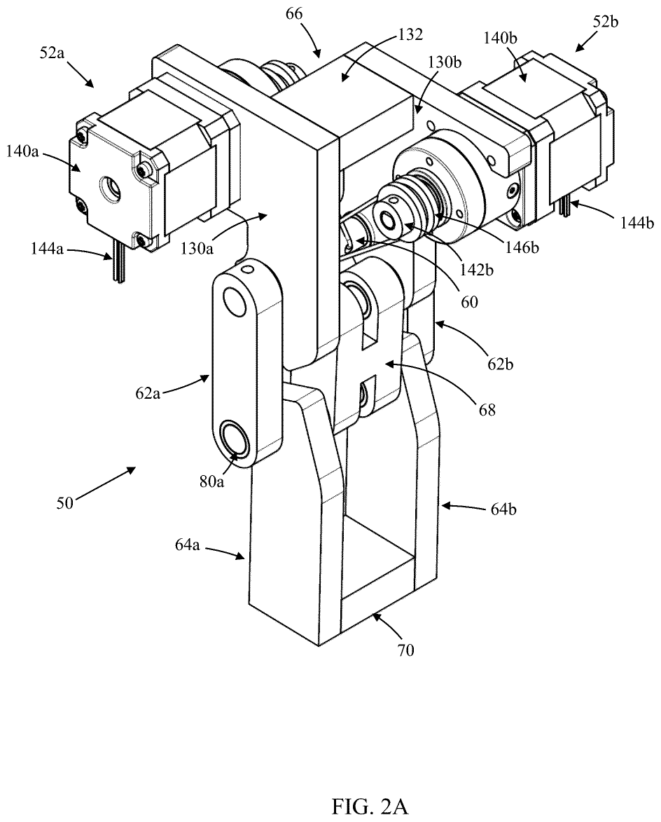

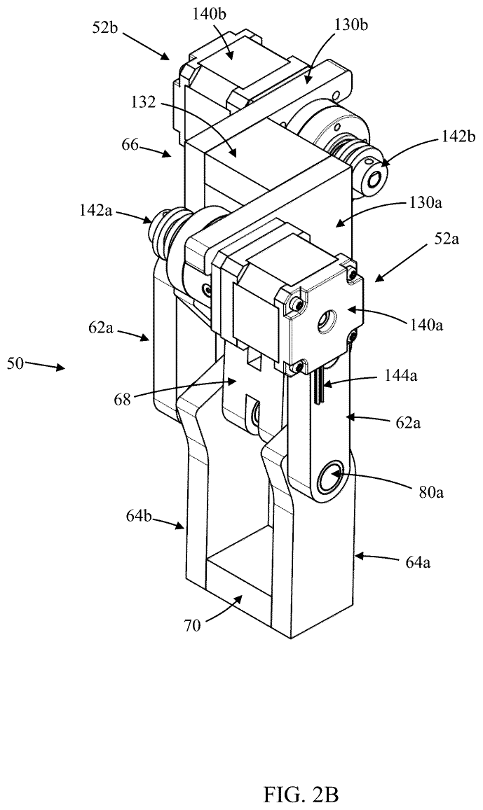

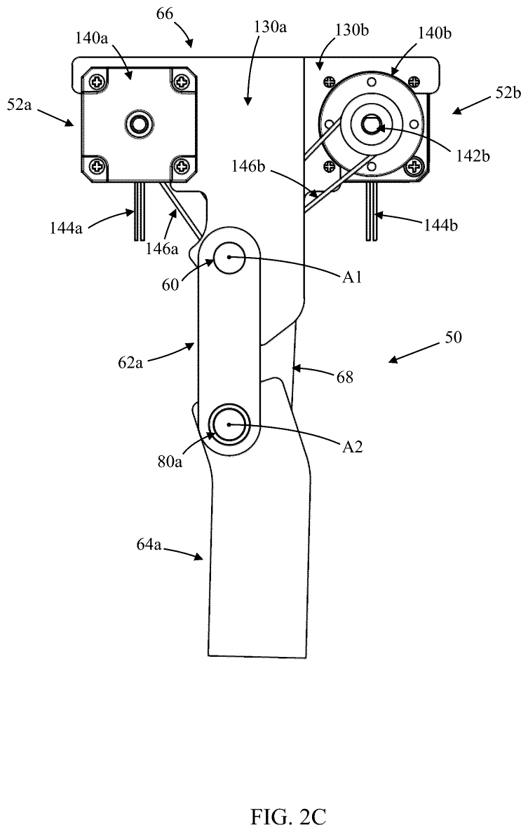

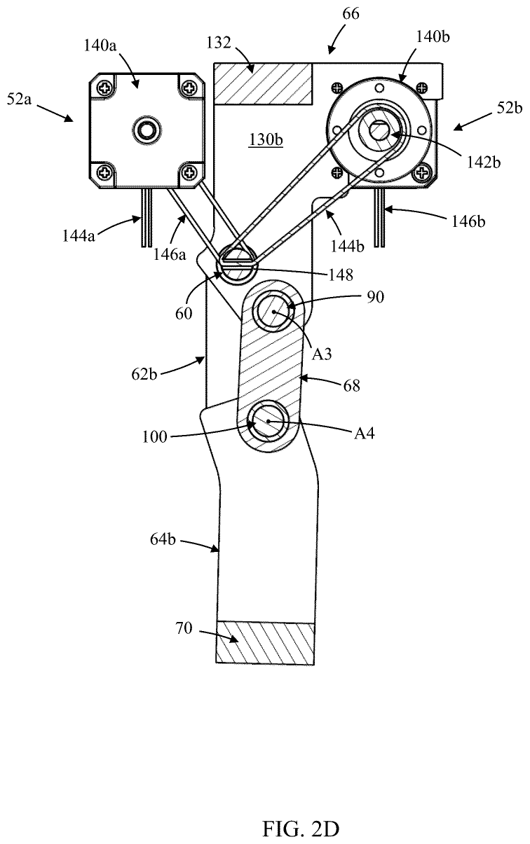

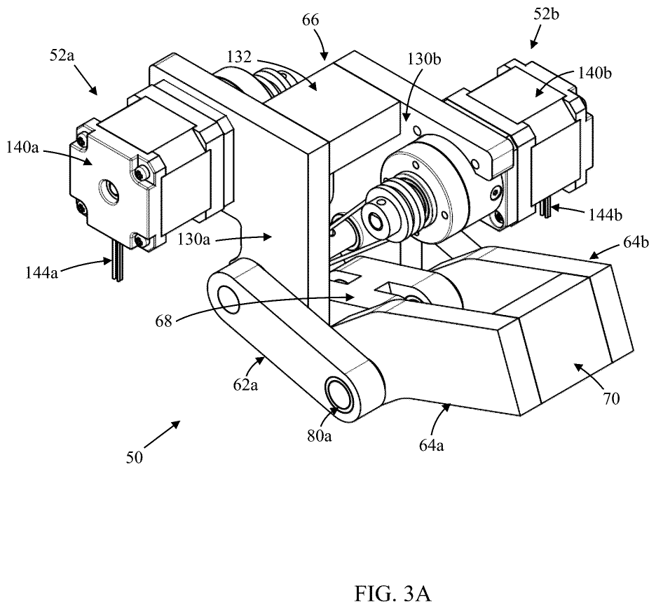

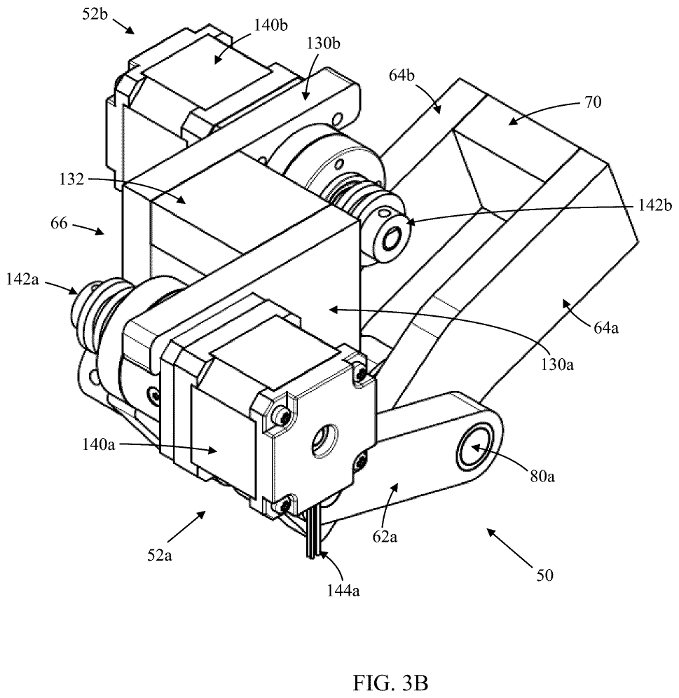

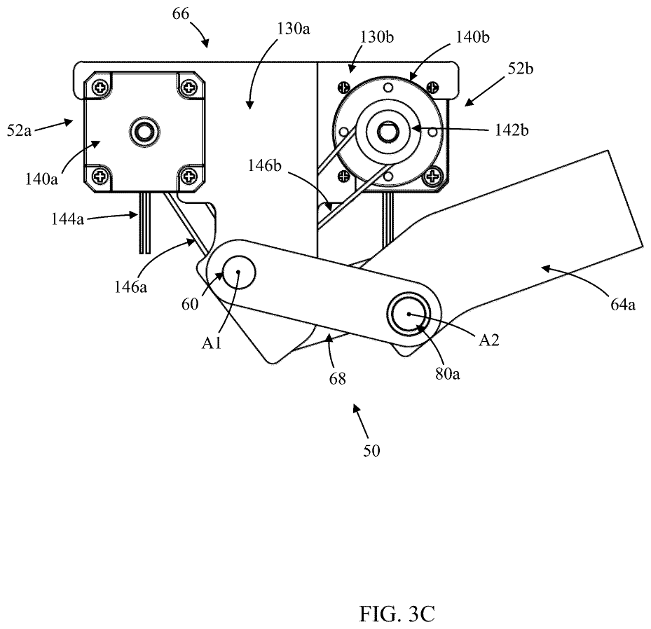

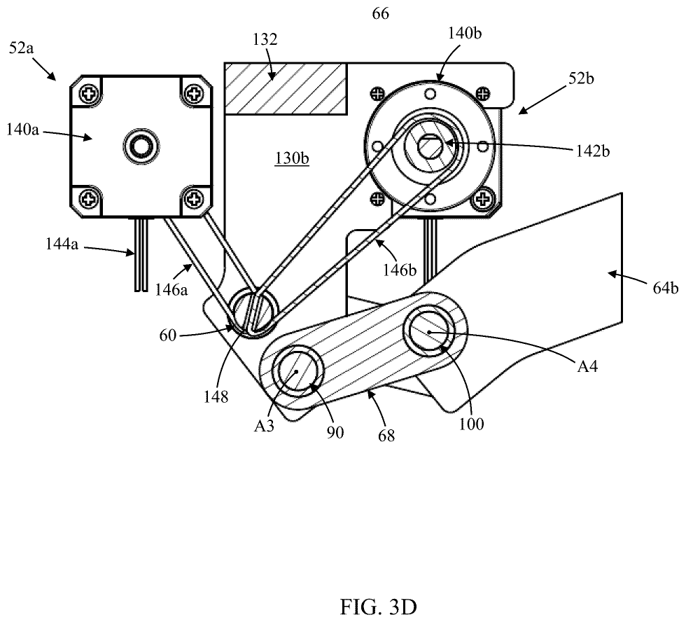

One example of the linkage assembly 50 in accordance with principles of the present disclosure is shown in greater detail in FIGS. 2A-2D and 3A-3D. As a point of reference, FIGS. 2A-2D illustrate the linkage assembly 50 in an “upright” arrangement (e.g., as generally reflected by FIGS. 1A and 1B in which the linkage assembly 50 arranges the prosthetic lower leg assembly 32 to be generally in line with the socket 30, and thus the residual thigh T); FIGS. 3A-3D illustrate the linkage assembly 50 in a “bent” arrangement. Stated otherwise, the linkage assembly 50 functions much like a normal human knee joint, articulating between a straightened leg (the upright or straight arrangement of FIGS. 2A-2D) and a bent leg (the bent arrangement of FIGS. 3A-3D). Further, with the examples of FIGS. 2A-2D and 3A-3D, two of the motors (labeled as 52a, 52b) are provided and serve as inputs to the linkage assembly 50.

The linkage assembly 50 includes a driven shaft 60, a first link (e.g., defined by a pair of first links members 62a, 62b), a second link (e.g., defined by a pair of second links 64a, 64b members), a base 66, an intermediate arm 68, and an output body 70. The driven shaft 60 is connected to a first end of each of the first links members 62a, 62b and is rotatably maintained by the base 66. The driven shaft 60 can be, or can be akin to, a cylindrical shaft and is rotatable relative to the base 66 about a first axis of rotation A1. An opposing, second end of each of the first link members 62a, 62b is rotatably connected to a first end of a respective one of the second link members 64a, 64b by a first shaft 80a, 80b that serves as a joint or second axis of rotation A2. The first link members 62a, 62b can be substantially identical in terms of size and shape (i.e., within 10 percent of truly identical size and shape), and the first shafts 80a, 80b are spatially aligned to in defining the common second axis of rotation A2 between the first link members 62a, 62b and the second link members 64a, 64b. An opposing second end of each of the second link members 64a, 64b is secured to the output body 70. The second link members 64a, 64b can be substantially identical in terms of size and shape (i.e., within 10 percent of truly identical size and shape), and are commonly connected to, and aligned with, the output body 70. A first end of the intermediate arm 68 is rotatably secured to the base 66 by a second shaft 90 (e.g., two shaft segments, one of which is best seen in FIGS. 2D and 3D) that serves as a joint or third axis of rotation A3. An opposing second end of the intermediate arm 68 is rotatably secured to each of the second link members 64a, 64b proximate the first end thereof by a third shaft 100 (e.g., two shaft segments, one of which is best seen in FIGS. 2D and 3D) that serves as a joint or fourth axis of rotation A4. The axes of rotation A1, A2, A3, and A4 are spatially off-set from one another, including the third axis of rotation A3 defined between the first link members 62a, 62b and the second link members 64a, 64b being spatially off-set form the fourth axis of rotation A4 defined between the second link members 64a, 64b and the intermediate arm 68. A geometry and size of the link members 62a, 62b, 64a, 64b, the intermediate arm 68 and a location of the each of the axes of rotation relative to one another is such that rotation of the driven shaft 60 is transmitted to the output body 70, causing the output body 70 to spatially articulate relative to the base 66 in a motion mimicking that of a human knee.

The base 66 can have various constructions, and in some embodiments forms or carry opposing, first and second plates 130a, 130b and a platform 132. The plates 130a, 130b can have a substantially identical construction (i.e., within 10 of truly identical size and shape) and are generally configured to rotatably receive and maintain the driven shaft 60 and the second shaft 120. A lateral spacing between the plates 130a, 130b is sufficiently sized to receive and permit free movement of the intermediate arm 68. Further, the plate(s) 130a, 130b are configured to support at least one motor module. With the non-limiting example of FIGS. 2A-2D, 3A-3D, the prosthetic knee unit 34 includes first and second motor modules 52a, 52b, with the first motor module 52a maintained by the first plate 130a, and the second motor module 52b maintained by the second plate 130b. The motor modules 52a, 52b are described in greater detail below. Other configurations are also acceptable. Regardless, the platform 132 extends between and interconnects the plates 130a, 130b, and can form or carry features appropriate for assembly to, or to serve as, the socket 30 (FIGS. 1A and 1B). Thus, upon final assembly and securement to a user, the platform 132 will be immediately proximate the residual thigh T (FIGS. 1A and 1B).

As best seen in FIGS. 2D and 3D, the motor modules 52a, 52b each include a motor 140a, 140b (e.g., a stepping motor) operable to rotate an output shaft 142a, 142b. The motors 140a, 140b are each electronically connectable to the controller 54 by wiring 144a, 144b. Further, the motor modules 52a, 52b each include a connection device for connecting the corresponding output shaft 142a, 142b with the driven shaft 60. With the non-limiting examples of FIGS. 2A-2D and 3A-3D, a belt 146a, 146b is provided with each of the modules 52a, 52b that connects the corresponding output shaft 142a, 142b with the driven shaft 60. Various constructions can be employed to secure each of the belts 146a, 146b with the driven shaft 60. For example, center bores 148 (one of which is visible in FIGS. 2D and 3D) can be formed through the driven shaft 60, each sized to receive a segment of a corresponding one of the belts 146a, 146b. Other assembly techniques are equally acceptable. Regardless, each of the belts 146a, 146b is arranged around the corresponding output shaft 142a, 142b and routed to the driven shaft 60. The output shafts 142a, 142b can be positioned at opposite sides of the first axis of rotation A1 defined by the driven shaft 60. With this construction, the motors 140a, 140b collectively occupy a relatively small footprint, but can operate in tandem generate desired rotational forces at the driven shaft 60 to cause desired articulation of the linkage assembly (e.g., serving an actuator function or operation) and to resist rotational forces being applied to the driven shaft 60 via the linkage assembly 60 (e.g., serving a damper function or operation), for example by each of the motors 140a, 140b being operable to rotate the corresponding output shaft 142a, 142b in both directions (clockwise and counterclockwise). In other embodiments, only a single motor module is provided.

The output body 70 can assume various configurations, and in some embodiments forms or carries features appropriate for assembly to the prosthetic lower leg assembly 32 (FIGS. 1A and 1B). Thus, upon final assembly and securement to a user, the output body 70 will be opposite or distal the residual thigh T (FIGS. 1A and 1B) as compared to the platform 132.

Generally, the linkage assembly 50 can be affected by operation of the motors 140a, 140b in a manner replicating the movement of a natural, healthy, adult human knee (i.e., a “gate cycle”). The linkage assembly 50 is configured to replicate the interaction of the femoral condyles rotating and sliding on the tibial plateaus of a natural human knee during a gait cycle according to various examples of the disclosure. An example of one gait cycle can include: 1) Heel Strike Phase (knee is fully or substantially fully extended), actuator is fully extended and damper is mid resistance to prepare for weight distribution and keep the knee feeling strong and in the extended position; 2) Stance Phase (knee is fully or substantially fully extended), actuator is fully extended and damper is at highest resistivity to keep knee extended to not bend; 3) Mid Swing Phase (knee is fully or substantially bent), actuator is fully contracted this enables toe clearance not matter what speed the person is moving and damper is at zero resistivity to allow for easy and smooth knee bend since there is no weight on the knee/leg; 4) Back to Heel Strike Phase as outlined above for repetition of Phases 1-3.

In one example, for the starting position at the beginning of a gate cycle, the linkage assembly 50 is designed to carry the center of gravity straight down through the center of axis of the femoral link; in this position, the linkage assembly 50 will not collapse. Once the center of gravity of the person shifts posteriorly or anteriorly, the linkage assembly 50 will flex and extend accordingly. Absent operation of the motor modules 52a, 52b, this can be performed when the user swings their residual limb and the momentum created is enough to move the linkage assembly 50 through its path of motion. Absent operation of the motor modules 52a, 52b, the user generally cannot stand on their other leg, lift the amputated leg and flex their prosthetic limb. Therefore, operation of the motor module(s) 52a as a damper and/or an actuator 80b is also configured to aid in helping to control the extension and flexion of the prosthetic knee unit 34. To get the system out of the dead lock position a user can move the force more posteriorly, when flexing the prosthetic knee unit 34. The damper will be at the highest resistivity during stance phase. In an actuator mode, the motor module(s) 52a, 52b helps the linkage assembly 50 to flex. In one example, the motor module(s) 52, 52b, operating in an actuator mode, will retract and lift the lower portion of the linkage assembly 50 to aid in flexion of the prosthetic knee unit 34.

Returning to FIG. 1, operation of the motor module(s) 52 is dictated by the controller 54 utilizing programming that references information provided by the one or more sensor 56. In some embodiments, the controller 54 is, or includes, a microprocessor, but can assume any computing-type processor format. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. The controller 54 may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer. Whether such elements are implemented as hardware or software depends upon the particular application and design constraints imposed on the overall system. Regardless, the controller 52 can be retained or worn by a user in various fashions, such as via a strap 160.

The sensor(s) 56 can assume various forms, and in some embodiments is an inertial measurement unit (“IMU”). It is envisioned that the IMU may comprise an accelerometer and/or a gyroscope, and be capable of detecting movement by a subject. In some aspects, the accelerometer and/or the gyroscope component may be capable of detecting motion and/or orientation along three axes. The IMU may be configured to generate data based on the detected position and or movement of the human subject. Such positional and/or movement data may comprise, e.g., a signal or data indicative of the degree, magnitude, speed or direction of motion of the subject's residual thigh T (e.g., anterior and/or posterior), lower back, or any other body part (or one or more portions thereof), and/or orientation data for the subject. In some aspects, absolute or relative timing parameters for any detected motion is derived from positional and/or movement data. In some aspects, an AC component of the IMU data provides movement data and the DC components provide subject orientation data.

Data obtained by the sensor(s) 56 can be provided to the controller 54 in various fashions. In some examples, the sensor(s) 56 wirelessly signal data to the controller 54. In other embodiments, a wired connection can be provided. Regardless, programming, modeling, and/or algorithms operated by the controller 54 determine or predict a state or activity of the user. Further, programming, modeling and/or algorithms operated by the controller determine or predict actuator and/or dampening forces to be applied by the motor module(s) 52 on the linkage assembly 50 to better meet the needs of the particular state or activity. In this regard, the programming algorithms can be tailored to an individual user (e.g., based on height, weight, etc.). For example, the user engaged in a walking activity can be determined or predicted by controller 54 based upon data from the sensor(s) 56, with the controller 54 then operating the motor module(s) 52 to assist the linkage assembly 50 in articulating through the gait cycle in a predetermined fashion.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

What is claimed is:

1. A prosthetic knee unit comprising:
a linkage assembly including a base, a driven shaft, at least one link, and an output body;
at least one motor module operably connected to the driven shaft;
a controller programmed to prompt operation of the at least one motor module;
wherein the prosthetic knee unit is configured such that the operation of the at least one motor module, as prompted by the controller, articulates the output body relative to the base via pivoting movement of the at least one link.

2. The prosthetic knee unit of claim 1, wherein the at least one link includes a first link and a second link, wherein a first end of the first link is secured to the driven shaft, a second end of the first link is rotatably coupled to a first end of the second link, and a send end of the second link is attached to the output body.

3. The prosthetic knee unit of claim 2, wherein the at least one link further includes an intermediate link, wherein a first end of the intermediate link is rotatably coupled to the base, and a second of the intermediate link is rotatably coupled to the second link.

4. The prosthetic knee unit of claim 3, wherein the driven shaft defines a first axis of rotation, a coupling between the first and second links defines a second axis of rotation, a coupling between the intermediate link and the base defines a third axis of rotation, and a coupling between the intermediate link and the second link defines a fourth axis of rotation, and further wherein the first, second, third, and fourth axes of rotation are offset from one another.

5. The prosthetic knee unit of claim 1, wherein the at least one motor module includes a first motor module and a second motor module, and further wherein each of the motor modules includes a motor rotatably driving an output shaft, and a belt connecting the output shaft to the driven shaft.

6. The prosthetic knee unit of claim 5, wherein the belt of each of the motor modules passes through a corresponding bore defined through the driven shaft.

7. The prosthetic knee unit of claim 5, wherein the motor of the first motor module and the motor of the second motor module are secured to the base.

8. The prosthetic knee unit of claim 1, further comprising at least one inertial measurement unit (IMU) configured for securement to a user, and further wherein data from the at least one IMU is signaled to the controller.

9. The prosthetic knee unit of claim 8, wherein the IMU includes at least one of a gyroscope and an accelerometer.

10. The prosthetic knee unit of claim 8, wherein the controller is programmed to control operation of the at least one motor module based upon information from the IMU.

11. The prosthetic knee unit of claim 10, wherein the controller is a microcontroller configured to be worn by a user.

12. The prosthetic knee unit of claim 10, wherein the controller is further programmed to prompt operation of the at least one motor module to serve as an actuator and as a damper based upon the determined activity state.

13. The prosthetic knee unit of claim 1, wherein the base includes a platform configured for securement a socket of a prosthetic leg.

14. The prosthetic knee unit of claim 1, wherein the output body is configured for securement to a prosthetic lower leg assembly of a prosthetic leg.

15. A prosthetic leg comprising:
a socket configured for securement to a residual thigh of a user;
a prosthetic knee unit of claim 1; and
a prosthetic lower leg assembly;
wherein the base is connected to the socket and the output body is connected to the prosthetic lower leg assembly.
